(19) United States
(12) Patent Application Publication
(10) Pub. No.: US 2002/0131948 A1
(43) Pub. Date: Sep. 19, 2002

(54) Tightening agent comprising at least one grafted silicone polymer

(75) Inventors: Beatrice Toumi, La Garenne Colombes (FR); Jean-Claude Garson, Suresnes (FR); Nathalie Mougin, Paris (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: L'OREAL, Paris (FR)

(21) Appl. No.: 10/060,244

(22) Filed: Feb. 1, 2002

Related U.S. Application Data
(63) Continuation of application No. 09/533,361, filed on Mar. 22, 2000.

(30) Foreign Application Priority Data
Mar. 22, 1999 (FR) 9903531

(51) Int. Cl. A61K 7/06; A61K 7/11

(52) U.S. Cl. 424/70.12; 424/401

(57) ABSTRACT

A composition useful as a tightening agent for decreasing or removing signs of aging, particularly for reducing or removing wrinkles and/or fine lines or to smooth out human skin. This composition comprises at least one grafted silicone polymer which itself comprises a polysiloxane portion and a portion composed of a non-silicone organic chain, one of the two portions constitutes the main chain of the polymer and the other is grafted to said main chain. Processes for the cosmetic treatment of aged skin, in particular wrinkled skin, comprising the application of an effective amount of the composition as defined above to achieve improvement in the appearance of rough, damaged or aged skin, or achieve softening the wrinkle by a tightening effect.

### BACKGROUND OF THE INVENTION

Field of the Invention:

The present invention relates to the use as tightening agent, in and/or for the manufacture of a composition intended to decrease or remove signs of aging, in particular to decrease or remove wrinkles and/or fine lines and/or to smooth out human skin, of at least one grafted silicone polymer comprising a polysiloxane portion and a portion composed of a non-silicone organic chain, one of the two portions constituting the main chain of the polymer and the other being grafted to the said main chain.

During the aging process, various signs appear on the skin which are highly characteristic of this aging, reflected in particular by a modification in the cutaneous structure and functions. The main signs of cutaneous aging are in particular the appearance of deep wrinkles and fine lines, which increase with age. In particular, a disruption of the “grain” of the skin is found, that is to say that the microrelief is less uniform and exhibits an anisotropic nature.

It is known to reduce these signs of aging by using cosmetic or dermatological compositions comprising active principles capable of combating aging, such as α-hydroxy acids, β-hydroxy acids and retinoids. These active principles act on wrinkles, in particular by removing the dead cells from the skin and by accelerating the process of cell replacement. However, these active principles exhibit the disadvantage of being effective in the treatment of wrinkles only after a certain application time. In point of fact, there is an increasing desire to obtain an immediate effect with the active principles used, resulting rapidly in the smoothing out of wrinkles and fine lines and in the disappearance of signs of fatigue.

Various compositions with a tightening effect which make it possible to obtain an immediate smoothing out of the skin have therefore been provided in the prior art.

These compositions generally comprise, as tightening agent, substances of natural origin, such as plant, egg, milk or animal derivatives. Thus, FR-A-2,758,083 discloses the use as tightener of a polymeric system having specific physicochemical properties which comprises at least one polymer of natural origin, such as a soya protein extract or chitin or keratin derivatives. In addition, Patent Application WO discloses firming compositions comprising a film-forming agent comprising at least one plant polysaccharide and hydrolysed casein as tightening active principles. However, the use of substances of natural origin is limited by the risks of bovine spongiform encephalopathy. Furthermore, apart from not always being reproducible, the tightening effect which these substances provide is not very great qualitatively and is of low persistence quantitatively.

Other compositions with a tightening effect of the prior art use synthetic polymers. Thus, a composition with a tightening effect comprising an aqueous dispersion of a polymeric system comprising at least one polymer of synthetic origin, chosen from various types of polyurethanes, from polyureas, from acrylic polymers or copolymers, from sulphonated isophthalic acid polymers and from their mixtures, is known from FR-A-2,758,084. The cosmetic feel of these compositions is not, however, always satisfactory.

Consequently, the need still remains for compounds which offer an immediate, sufficient and lasting tightening effect without risk to the consumer.

A subject-matter of the present invention is therefore the use of a grafted silicone polymer which makes it possible to obtain this effect.

More particularly, a subject-matter of the present invention is the use, in a cosmetic composition intended to decrease or remove signs of cutaneous aging and more particularly to reduce or remove wrinkles and/or fine lines of the skin and/or to smooth out the skin, of at least one grafted silicone polymer comprising a polysiloxane portion and a portion composed of a non-silicone organic chain, one of the two portions constituting the main chain of the polymer and the other being grafted to the said main chain.

Another subject-matter of the invention is the use as tightening agent, in a cosmetic composition, of at least one grafted silicone polymer comprising a polysiloxane portion and a portion composed of a non-silicone organic chain, one of the two portions constituting the main chain of the polymer and the other being grafted to the said main chain.

In the context of this description and the appended claims, the term “tightening agent” is understood to mean compounds capable of having an apparent tightening effect, that is to say of smoothing out the skin and immediately reducing, indeed bringing about the disappearance of, wrinkles and fine lines.

A further subject-matter of the invention is the use, in the manufacture of a composition intended to decrease or remove signs of cutaneous aging, in particular to reduce the appearance of rough, damaged or aged skin, reduce or remove wrinkles and/or fine lines of the skin and/or to smooth out the skin, of at least one grafted silicone polymer comprising a polysiloxane portion and a portion composed of a non-silicone organic chain, one of the two portions constituting the main chain of the polymer and the other being grafted to the said main chain.

The composition used in the present invention comprises, in addition to the above-mentioned grafted silicone polymer, a physiologically acceptable medium, that is to say a medium compatible with the skin and its superficial body growths, the mucous membranes and the semimucous membranes.

The amount of grafted silicone polymer present in the composition depends on the tightening effect which it is desired to confer on this composition and it generally represents from 0.03 to 25% and preferably from 0.3 to 6%, better still approximately 2%, of the total weight of the composition.

The present invention also relates to a process for the cosmetic treatment of aged skin, in particular wrinkled skin, comprising the application to the said skin of at least one grafted silicone polymer as mentioned above in an amount which is effective in softening the wrinkle by a tightening effect.

The grafted silicone polymers mentioned above are well known to a person skilled in the art. Thus, the use of anionic grafted silicone derivatives as fixing materials in an aerosol, such as a lacquer, for shaping and/or retaining the form of the hairstyle, is known from EP 848,941. These compounds are also used to improve the styling effect of detergent hair compositions comprising at least one cationic polymer, such as those disclosed in EP 756,860 and WO 97/46210.

As indicated above, the grafted silicone polymers according to the invention can be chosen from polymers with a non-silicone organic backbone grafted by at least one monomer comprising a polysiloxane, polymers with a polysiloxane backbone grafted by at least one non-silicone organic monomer, and their mixtures.

These two types of grafted silicone polymers will now be described in more detail. As a preliminary, the following definitions are given:

1. “silicone” or “polysiloxane”: in the context of this description, this term is understood to mean, in conformity with what is generally accepted, any organosilicon polymer or oligomer with a branched or crosslinked, linear or cyclic structure of variable molecular weight obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbonaceous radicals being directly bonded via a carbon atom to the said silicon atoms. The commonest hydrocarbonaceous radicals are alkyl radicals, especially C₁-C₁₀ alkyl radicals and in particular methyl radicals, fluoroalkyl radicals, aryl radicals and in particular phenyl radicals, and alkenyl radicals and in particular vinyl radicals; other types of radicals capable of being bonded either directly or via a hydrocarbonaceous radical to the siloxane chain are in particular hydrogen, halogens and in particular chlorine, bromine or fluorine, thiols, alkoxy radicals, polyoxyalkylene (or polyether) radicals and in particular polyoxyethylene and/or polyoxypropylene radicals, hydroxyl or hydroxyalkyl radicals, amino groups, which may or may not be substituted, amide groups, acyloxy or acyloxyalkyl radicals, hydroxyalkylamino or aminoalkyl radicals, quaternary ammonium groups, amphoteric or betaine groups, or anionic groups, such as carboxylate, thioglycolate, sulphosuccinate, thiosulphate, phosphate and sulphate groups, this list, of course, being in no way limiting (so-called “organomodified” silicones).

2. “polysiloxane macromer”: in conformity with what is generally accepted, this expression denotes, in the present description, any monomer comprising, in its structure, a polymer chain of the polysiloxane type.

Polymers with a Non-Silicone Organic Backbone Grafted by at Least One Monomer Comprising a Polysiloxane

The polymers with a non-silicone organic backbone grafted by monomers comprising a polysiloxane in accordance with the present invention are composed of an organic main chain formed from organic monomers not comprising silicone, to which is grafted, within the said chain and optionally at at least one of its ends, at least one polysiloxane macromer.

The non-silicone organic monomers constituting the main chain of the grafted silicone polymer can be chosen from monomers with ethylenic unsaturation polymerizable by the radical route, monomers polymerizable by polycondensation, such as those forming polyamides, polyesters or polyurethanes, or monomers with ring opening, such as those of the oxazoline or caprolactone type. Non-silicone organic monomers with an anionic nature will advantageously be chosen.

The polymers with a non-silicone organic backbone grafted by monomers comprising a polysiloxane in accordance with the present invention can be obtained by any means known to a person skilled in the art, in particular by reaction between (i) a starting polysiloxane macromer correctly functionalized on the polysiloxane chain and (ii) one or a number of non-silicone organic compounds, themselves correctly functionalized by a functional group which is capable of reacting with the functional group or groups carried by the said macromer with the formation of a covalent bond; a classic example of such a reaction is the radical reaction between a vinyl group carried on one of the ends of the macromer and a double bond of a monomer with ethylenic unsaturation of the main chain.

The polymers with a non-silicone organic backbone grafted by monomers comprising a polysiloxane in accordance with the invention are preferably copolymers obtained by radical polymerization from monomers with ethylenic unsaturation and from silicone macromers having an end vinyl group or alternatively copolymers obtained by reaction of a polyolefin comprising functionalized groups and of a polysiloxane macromer having an end functional group which is reactive with the said functionalized groups.

A specific family of grafted silicone polymers which is suitable for use in the present invention is composed of the grafted silicone copolymers comprising:

a) at least one lipophilic monomer (A) with ethylenic unsaturation which is polymerizable by the radical route;

b) at least one polar hydrophilic monomer (B) with ethylenic unsaturation which is copolymerizable with the monomer or monomers of the (A) type such that (A) +(B) varies from 99.99% to 50% by weight; and

c) from 0.01 to 50% by weight of at least one polysiloxane macromer (C) of general formula:

X(Y)ₙSi(R)₃₋ₘZₘ(I)

where:

X denotes a group with ethylenic unsaturation which is copolymerizable with the monomers (A) and (B);

Y denotes a group with divalent bonding;

R denotes a hydrogen, a hydroxyl group, a C₁-C₆ alkyl or alkylamino or alkoxy group or a C₆-C₁₂ aryl group;

Z denotes a polysiloxane unit having a number-average molecular weight of at least 50;

n is 0 or 1 and m is an integer ranging from 1 to 3;

the percentages being calculated with respect to the total weight of the monomers (A), (B) and (C).

These polymers, and processes for the preparation thereof, are disclosed in patents U.S. Pat. Nos. 4,693,935, 4,728,571 and 4,972,037 and Patent Applications EP-A-0,412,704, EP-A-0,412,707 and EP-A-0,640,105. They have a number-average molecular weight preferably ranging from 10,000 to 2,000,000 and preferably a glass transition temperature Tg or a crystalline melting temperature Tm of at least −20° C.

Mention may be made, as examples of lipophilic monomers (A), of esters of acrylic or methacrylic acid with C₁-C₁₈ alcohols; styrene; polystyrene macromers; vinyl acetate; vinyl propionate; α-methylstyrene; tert-butylstyrene; butadiene; cyclohexadiene; ethylene; propylene; vinyltoluene; esters of acrylic or methacrylic acid with 1,1-dihydroperfluoroalkanol or with its homologues; esters of acrylic or methacrylic acid with ω-hydridofluoroalkanol; esters of acrylic or methacrylic acid with fluoroalkylsulphonamidoalcohol; esters of acrylic or methacrylic acid with fluoroalkyl alcohol; esters of acrylic or methacrylic acid with alcohol fluoroether; or their mixtures.

The preferred monomers (A) are chosen from the group composed of n-butyl methacrylate, isobutyl methacrylate, tert-butyl acrylate, tert-butyl methacrylate, 2-ethylhexyl methacrylate, methyl methacrylate, 2-(N-methylperfluorooctanesulphon-amido)ethyl acrylate, 2-(N-butylperfluorooctanesulphonamido)ethyl acrylate and their mixtures.

Mention may be made, as examples of polar monomers (B), of acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and their half-esters, hydroxyalkyl (meth)acrylates, diallyldimethylammonium chloride, vinylpyrrolidone, vinyl ethers, maleimides, vinylpyridine, vinylimidazole, polar heterocyclic vinyl compounds, styrenesulphonate, allyl alcohol, vinyl alcohol, vinylcaprolactam or their mixtures.

The preferred monomers (B) are chosen from the group composed of acrylic acid, N,N-dimethylacrylamide, dimethylaminoethyl methacrylate, quaternized dimethylaminoethyl methacrylate, vinylpyrrolidone and their mixtures.

The preferred polysiloxane macromers (C) of formula (I) are chosen from those corresponding to the following general formula (II):

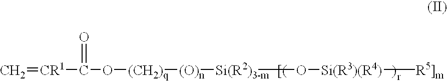

in which:

R¹ is hydrogen or methyl;

R² is C₁-C₆ alkyl, alkoxy or alkylamino, C₆-C₁₂ aryl or hydroxyl;

R³ is C₁-C₆ alkyl, alkoxy or alkylamino, C₆-C₁₂ aryl or hydroxyl;

R⁴ is C₁-C₆ alkyl, alkoxy or alkylamino, C₆-C₁₂ aryl or hydroxyl;

R⁵ is C₁-C₆ alkyl, alkoxy or alkylamino, C₆-C₁₂ aryl or hydroxyl;

q is an integer from 2 to 6;

n has the value 0 or 1;

r is an integer from 0 to 750; and

m is an integer ranging from 1 to 3.

Use is more particularly made of the polysiloxane macromers of formula (III):

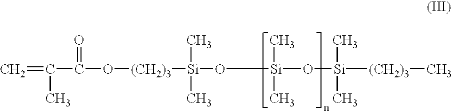

with n being a number ranging from 1 to 700.

A particularly preferred embodiment of the invention consists in using, as grafted silicone polymer, a copolymer capable of being obtained by radical polymerization from the mixture of monomers consisting of:

a) 60 weight % of tert-butyl acrylate;

b) 20 weight % of acrylic acid;

c) 20 weight % of silicone macromer of formula (III):

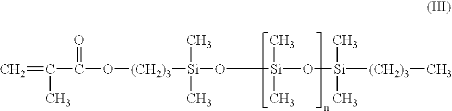

with n being a number ranging from 1 to 700, the percentages by weight being calculated with respect to the total weight of the monomers.

This copolymer will subsequently be denoted by “acrylic/acrylate copolymer grafted by PDMS methacrylate”.

Polymers with a Polysiloxane Backbone Grafted by at Least One Non-Silicone Organic

According to the present invention, the grafted silicone polymer or polymers with a polysiloxane backbone grafted by non-silicone organic monomers comprise a main silicone (or polysiloxane (≡Si—O—)ₙ) chain to which is grafted, within the said chain and optionally at at least one of its ends, at least one organic group not comprising silicone.

The polymers with a polysiloxane backbone grafted by non-silicone organic monomers according to the invention can be existing commercial products or alternatively be obtained according to any means known to a person skilled in the art, in particular by reaction between (i) a starting silicone correctly functionalized on one or a number of its silicon atoms and (ii) a non-silicone organic compound itself correctly functionalized by a functional group which is capable of reacting with the functional group or groups carried by the said silicone with the formation of a covalent bond; a classic example of such a reaction is the hydrosilylation reaction between ≡Si—H groups and CH₂═CH— vinyl groups or alternatively the reaction between —SH thio-functional groups and these same vinyl groups.

Examples of polymers with a polysiloxane backbone grafted by non-silicone organic monomers suitable for an implementation of the present invention, as well as their specific method of preparation, are in particular disclosed in Patent Applications EP-A-0,582,152, WO 93/23009 and WO 95/03776, the teachings of which are completely included within the present description by way of non-limiting references.

According to a particularly preferred embodiment of the present invention, the silicone polymer with a polysiloxane backbone grafted by non-silicone organic monomers used comprises the result of the radical copolymerization between, on the one hand, at least one anionic non-silicone organic monomer exhibiting an ethylenic unsaturation and/or one hydrophobic non-silicone organic monomer exhibiting an ethylenic unsaturation and, on the other hand, a silicone exhibiting, in its chain, at least one functional group capable of reacting with the said ethylenic unsaturations of the said non-silicone monomers with the formation of a covalent bond, in particular thio-functional groups.

According to the present invention, the said anionic monomers with ethylenic unsaturation are preferably chosen, alone or as mixtures, from linear or branched unsaturated carboxylic acids, optionally partially or completely neutralized in the form of a salt, it being possible for this or these unsaturated carboxylic acids to be more particularly acrylic acid, methacrylic acid, maleic acid, maleic anhydride, itaconic acid, fumaric acid and crotonic acid. Suitable salts are in particular the alkali metal, alkaline earth metal and ammonium salts. It should be noted that, likewise, in the final grafted silicone polymer, the organic group of anionic nature which the result of the radical (homo)polymerization of at least one anionic monomer of unsaturated carboxylic acid type comprises can, after reaction, be post-neutralized with a base (sodium hydroxide, ammonia, and the like) in order to convert it to the form of a salt.

According to the present invention, the hydrophobic monomers with ethylenic unsaturation are preferably chosen, alone or as mixtures, from alkanol acrylic acid esters and/or alkanol methacrylic acid esters. The alkanols are preferably C₁-C₁₈ alkanols and more particularly C₁-C₁₂ alkanols. The preferred monomers are chosen from the group composed of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate or their mixtures.

A family of silicone polymers with a polysiloxane backbone grafted by non-silicone organic monomers which is particularly well suited to the implementation of the present invention is composed of silicone polymers comprising, in their structure, the following unit of formula (IV):

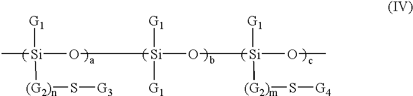

in which the G₁ radicals, which are identical or different, represent hydrogen or a C₁-C₁₀ alkyl radical or alternatively a phenyl radical; the G₂ radicals, which are identical or different, represent a C₁-C₁₀ alkylene group; G₃ represents a polymer residue resulting from the (homo)polymerization of at least one anionic monomer with ethylenic unsaturation; G₄ represents a polymer residue resulting from the (homo)polymerization of at least one hydrophobic monomer with ethylenic unsaturation; m and n are, independently of one another, equal to 0 or 1; a is an integer ranging from 0 and 50; b is an integer which can be between 10 and 350 and c is an integer ranging from 0 and 50, with the proviso that one of the parameters a and c is other than 0.

The above unit of formula (IV) preferably exhibits at least one, and more preferably still all, of the following characteristics:

the G₁ radicals denote a C₁-C₁₀ alkyl radical;

n is not zero and the G₂ radicals represent a divalent C₁-C₃ radical;

G₃ represents a polymer radical resulting from the (homo)polymerization of at least one monomer of the carboxylic acid with ethylenic unsaturation type, preferably acrylic acid and/or methacrylic acid;

G₄ represents a polymer radical resulting from the (homo)polymerization of at least one monomer of the (C₁-C₁₀)alkyl (meth)acrylate type.

Examples of grafted silicone polymers corresponding to the formula (IV) are thus in particular polydimethylsiloxanes (PDMS) to which are grafted, via a connecting link of thiopropylene type, mixed polymer units of the poly((meth)acrylic acid) type and of the poly(alkyl (meth)acrylate) type.

These polymers are referenced as “polysilicone-8” according to the CTFA nomenclature.

They include, for instance, a propylthio (polymethyl acrylate), propylthio (polymethyl methacrylate) and propylthio (polymethacrylic acid) grafted polydimethylsiloxane. Alternatively, they can include a (polyisobutyl methacrylate) and propylthio (polymethacrylic acid) grafted polydimethylsiloxane.

Such grafted silicone polymers are sold in particular by the Company 3M under the trade names VS70, VS80 and LO21.

The number-average molecular mass of the silicone polymers with a polysiloxane backbone grafted by non-silicone organic monomers of the invention preferably varies from 10,000 to 1,000,000 approximately and more preferably still from 10,000 to 100,000 approximately.

The composition of the invention can be provided in all the pharmaceutical dosage forms normally used for a topical application on the skin, in particular in the form of an aqueous, aqueous/alcoholic or oily solution, of an oil-in-water or water-in-oil or multiple emulsion, of an aqueous or oily gel, of a liquid, pasty or solid anhydrous product, or of a dispersion of oil in an aqueous phase in the presence of spherules, it being possible for these spherules to be polymeric nanoparticles, such as nanospheres and nanocapsules, or better still lipid vesicles of ionic and/or non-ionic type.

This composition can be more or less fluid and have the appearance of a white or coloured cream, of an ointment, of a milk, of a lotion, of a serum, of a paste or of a foam. It can optionally be applied to the skin in the form of an aerosol. It can also be provided in the solid form and, for example, in the form of a stick. It can be used as a product for caring for and/or as a product for making up the skin.

The composition of the invention more particularly constitutes an antiwrinkle composition which is provided in particular in the form of a serum.

In a known way, the composition of the invention can also comprise the adjuvants usual in the cosmetics field, such as hydrophilic or lipophilic gelling agents, hydrophilic or lipophilic active principles, preservatives, antioxidants, solvents, fragrances, fillers, screening agents, pigments, odour absorbers and colouring materials. The amounts of these various adjuvants are those conventionally used in the field under consideration, for example from 0.01 to 20% of the total weight of the composition. These adjuvants, depending on their nature, can be introduced into the fatty phase, into the aqueous phase, into the lipid vesicles and/or into the nanoparticles. These adjuvants and their concentrations must be such that they do not modify the desired tightening property of the polymeric system.

When the composition of the invention is an emulsion, the proportion of the fatty phase can range from 5 to 80% by weight and preferably from 5 to 50% by weight with respect to the total weight of the composition. The fatty substances, the emulsifiers and the coemulsifiers used in the composition in the form of an emulsion are chosen from those conventionally used in the field under consideration. The emulsifier and the coemulsifier are preferably present in the composition in a proportion ranging from 0.3 to 30% by weight and preferably from 0.5 to 20% by weight with respect to the total weight of the composition.

Mention may be made, as fatty substances which can be used in the invention, of oils and in particular mineral oils (liquid petrolatum), oils of vegetable origin (avocado oil, soybean oil), oils of animal origin (lanolin), synthetic oils (perhydrosqualene), silicone oils (cyclomethicone) and fluorinated oils (perfluoropolyethers). Use may also be made, as fatty substances, of fatty alcohols (cetyl alcohol), fatty acids, waxes and gums and in particular silicone gums.

Mention may be made, as emulsifiers and coemulsifiers which can be used in the invention, for example, of polyethylene glycol fatty acid esters, such as PEG-50 stearate and PEG40 stearate, and polyol fatty acid esters, such as glyceryl stearate and sorbitan tristearate.

Mention may in particular be made, as hydrophilic gelling agents, of carboxyvinyl polymers (carbomer), acrylic copolymers, such as acrylate/alkyl acrylate copolymers, polyacrylamides, polysaccharides, natural gums and clays and mention may be made, as lipophilic gelling agents, of modified clays, such as bentones, metal salts of fatty acids, hydrophobic silica and polyethylenes.

Use may in particular be made, as active principles, of polyols (glycerol, propylene glycol), vitamins, keratolytic and/or desquamating agents, soothing agents and their mixtures.

In addition, the tightening agents used according to the invention can also be used in combination with other compounds known to a person skilled in the art as tightening agents which have properties different from those of the agents used according to the invention, in particular a protein or a protein hydrolysate. Mention may be made, as compounds of this type, for example, of milk proteins, such as lactalbumin, plant proteins, such as the soya protein sold under the name of Eleseryl by the company LSN, the oats derivative sold under the name “Reductine” by the company Silab or nucleic acids, such as DNA. It is possible, as an alternative form, to combine the tightening agents according to the invention with the tightening polymers disclosed in FR-A-2,758,084.

Thus, the composition according to the invention preferably comprises, in a physiologically acceptable medium, at least one grafted silicone polymer as defined above and one or more plant products such as extracts, chemicals, enzymes or proteins. It may also comprise one or more products, extracts, chemicals, enzymes or proteins derived from animals, algae, fungi, yeast, or other eukaryotic or prokaryotic organisms.

As an alternative form or in addition, it is preferable for the composition according to the invention to comprise, in a physiologically acceptable medium, at least one grafted silicone polymer as defined above and one or more draining, lipolytic, disinfiltrating, slimming, firming, antiglycant and/or vasoprotective compounds.

Use may be made, as slimming active principles, in the composition of the invention of any compound known per se as exhibiting a more or less marked activity in the field of combating adiposity and/or cellulite and/or as slimming agents for the face. They can be chosen, for example, from:

1) phosphodiesterase inhibitors, such as:

xanthine derivatives, such as caffeine and its derivatives, in particular the 1-hydroxyalkylxanthines disclosed in the document FR-A-2,617,401, caffeine citrate, theophylline and its derivatives, theobromine, acefylline, aminophylline, chloroethyltheophylline, diprophylline, diniprophylline, etamiphylline and its derivatives, etofylline or proxyphylline;

combinations comprising xanthine derivatives, such as the combination of caffeine and silanol (methylsilanetriol derivative of caffeine), for example the product sold by the company Exsymol under the name cafeisilane C;

compounds of natural origin comprising xanthine bases and in particular caffeine, such as tea, coffee, guarana, mate or cola (Cola nitida) extracts and in particular the dry extract of guarana (Paulina sorbilis) fruit comprising 8 to 10% of caffeine;

ephedrine and its derivatives, which can in particular be found in the natural state in plants such as Ma Huang (Ephedra plant).

2) plant extracts and extracts of marine origin, which are active with regard to the receptors to be inhibited, such as α2-blockers or NPY-blockers, or active in stimulating β-receptors and proteins G, resulting in the activation of adenyl cyclase, or alternatively those which act on lipoprotein lipase, which inhibits the capturing of glucose, or which act on other enzymes which are favourable to lipolysis and/or are unfavourable to lipogenesis. Mention may be made, as plant extracts of this type, for example, of:

Garcinia cambogia,

Bupleurum chinensis extracts,

extracts of English ivy (Hedera helix), of mountain tobacco (Arnica montana L), of rosemary (Rosmarinus officinalis), of marigold (Calendula officinalis), of sage (Salvia officinalis L), of ginseng (Panax ginseng), of St John's Wort (Hypericum perforatum), of butcher's broom (Ruscus aculeatus L), of meadowsweet (Filipendula ulmaria L), of cat's whiskers (Orthosiphon stamineus Benth) or of birch (Betula alba),

Ginkgo biloba extracts,

horsetail extracts,

horse chestnut extracts,

cangzhu extracts,

Chrysanthellum indicum extracts,

extracts from plants of the genus Armeniacea, Atractylodis, Platicodon, Sinommenum, Pharbitidis or Flemingia,

Coleus extracts, such as C. forskohlii, C. blumei, C. esquirolii, C. scuttellaroides, C. xanthantus and C. barbatus extracts, such as the Coleus barbatus root extract comprising 60% of forskolin,

Ballota extracts,

extracts of Guioa, Davallia, Terminalia, Barringtonia, Trema or Antirobia.

Mention may be made, as extract of marine origin, of:

algal extracts, such as the red alga (Gelidium cartilagineum) extract sold by the company Secma under the trade name Rhodysterol or the Laminaria digitata extract sold under the name Phycox75 by the company Secma,

protamines and their derivatives, such as those disclosed in the document FR-A-2,758,724.

3) slimming active principles which bind to PPARs (Peroxisome Proliferator Activated Receptors), such as those disclosed in the documents WO-A-97/10813 and WO-96/33724, or active principles which block the differentiation of the precursor cells of adipocytes in adipose tissue.

The amount of slimming active principle(s) can vary to a large extent and depends on the nature of the active principle or principles used. The slimming active principle or principles are generally present at a concentration ranging from 0.05 to 20% and preferably from 0.1 to 10% by weight with respect to the total weight of the composition.

Active principles which will complement the action of the slimming active principles can be added to these slimming active principles, in particular:

1) active principles which act on the microcirculation (vasoprotector or vasodilator), such as flavonoids, ruscogenins, esculosides, the aescine extracted from horse chestnut, nicotinates, hesperidin methyl chalcone, butcher's broom, essential oils of lavender or rosemary, and some rutin derivatives, such as the disodium salt of rutinyl sulphate;

2) firming active principles and/or antiglycant active principles (which prevent sugar from attaching to collagen fibres), such as Centella asiatica and Siegesbeckia extracts and yeast extracts of the Saccharomyces cerevisiae type, which stimulate the synthesis of collagen, and also silicon and amadorine, which act as anti-glycant agents.

Thus, the composition according to the invention advantageously includes, in a physiologically acceptable medium, at least one grafted silicone polymer as defined above and one or more compounds chosen from: a horse chestnut extract, an ivy extract, a butcher's broom extract, a Bupleurum chinensis extract, an algal extract, caffeine and a rutin derivative.

The amount of these active principles can vary to a large extent. These active principles are generally present at a concentration ranging from 0.01 to 15% and preferably from 0.05 to 10% by weight with respect to the total weight of the composition.

In the event of incompatibility, the active principles indicated above can be incorporated in spherules, in particular ionic or non-ionic vesicles and/or nanoparticles (nanocapsules and/or nanospheres), so as to isolate them from one another in the composition.

The examples below of compositions according to the invention are given by way of illustration and without a limiting nature. The amounts are given therein as percentages by weight.

Example 1: Antiwrinkle cream

| Hydrogenated polyisobutene | | 5.5% |
|---|---|---|
| Isostearyl neopentanoate | | 3.5% |
| Glyceryl stearate and PEG-100 stearate | | 2.0% |
| Fatty alcohols | | 3.5% |
| Stearic acid | | 3.0% |
| Cyclomethicone | | 11.0% |
| Polyacrylamide and C₁₃-C₁₄ isoparaffin | | 1.7% |
| and laureth-7 (Sepigel 305, sold by the | | |
| company Seppic) | | |
| Polysilicone-8 (VS 80 from 3M), as a | | 7.0% |
| 30% aqueous solution | | |
| Preservatives | | 0.3% |
| Neutralizing agent | | 0.03% |
| Demineralized water | q.s. for | 100% |

Example 2: Antiageing serum

| Polyacrylamide and C₁₃-C₁₄ isoparaffin | | 1.0% |
|---|---|---|
| and laureth-7 (Sepigel 305, sold by the | | |
| company Seppic) | | |
| Xanthan gum | | 0.2% |
| Maleic anhydride and methyl vinyl ether | | 0.2% |
| polymer crosslinked by 1,9-decadiene | | |
| Triethanolamine | | 0.2% |
| Polysilicone-8 (VS 80 from 3M), as a | | 7.0% |
| 30% aqueous solution | | |
| Preservatives | | 0.3% |
| Demineralized water | q.s. for | 100% |

Example 3: Antiwrinkle cream

| Hydrogenated polyisobutene | | 5.5% |
|---|---|---|
| Isostearyl neopentanoate | | 3.5% |
| Glyceryl stearate and PEG-100 stearate | | 2.0% |
| Fatty alcohols | | 3.5% |
| Stearic acid | | 3.0% |
| Cyclomethicone | | 11.0% |
| Polyacrylamide and C₁₃-C₁₄ isoparaffin | | 1.7% |
| and laureth-7 (Sepigel 305, sold by the | | |
| company Seppic) | | |
| Acrylic/acrylate copolymer grafted by | | 7.0% |
| PDMS methacrylate | | |
| Preservatives | | 0.3% |
| Neutralizing agent | | 0.03% |
| Demineralized water | q.s. for | 100% |

Example 4: Antiageing serum

| Polyacrylamide and C₁₃-C₁₄ isoparaffin | | 1.0% |
|---|---|---|
| and laureth-7 (Sepigel 305, sold by the | | |
| company Seppic) | | |
| Xanthan gum | | 0.2% |
| Maleic anhydride and methyl vinyl ether | | 0.2% |
| polymer crosslinked by 1,9-decadiene | | |
| Triethanolamine | | 0.2% |
| Acrylic/acrylate copolymer grafted by | | 7.0% |
| PDMS methacrylate | | |
| Preservatives | | 0.3% |
| Demineralized water | q.s. for | 100% |

Example 5: Slimming cream for the face

| Hydrogenated polyisobutane | | 5.0% |
|---|---|---|
| Aescine | | 0.1% |
| Caffeine | | 0.3% |
| Glycerol and propylene glycol | | 7% |
| Cyclomethicone and dimethiconol | | 2.2% |
| Soya protein | | 5% |
| Disodium salt of rutinyl disulphate | | 0.1% |
| Gelling agent | | 1% |
| Polysilicone-8 (VS80 from 3M), as a | | 1% |
| 30% aqueous solution | | |
| Gelidium cartilagineum extract | | 2.5% |
| (Rhodysterol from Secma) | | |
| Bupleurum chinensis rhizome extract | | 2% |
| and caffeine (Pleurimincyl from Sederma) | | |
| Alcohol | | 15% |
| Fillers | | 0.2% |
| Neutralizing agent | | 0.013% |
| Preservatives | | 0.25% |
| Water | q.s. for | 100% |

Example 6: Shaping serum for the face

| Pentaerythrityl tetraoctanoate | | 5.0% |
|---|---|---|
| English ivy extract | | 0.1% |
| Caffeine | | 0.5% |
| Terminalia sericea extract | | 0.1% |
| Glycerol | | 3% |
| Cyclomethicone | | 2% |
| Soya protein | | 10% |
| Disodium salt of rutinyl disulphate | | 0.05% |
| Gelling agents | | 3% |
| Polysilicone-8 (VS80 from 3M), as a | | 1% |
| 30% aqueous solution | | |
| Yeast extract | | 3.5% |
| Alcohol | | 10% |
| Neutralizing agent | | 0.013% |
| Preservatives | | 0.25% |
| Water | q.s. for | 100% |

### EXAMPLE 7

### Sensory Evaluation of the Tightening Effect

The serum of Example 2 above was tested on a panel of five women exhibiting wrinkles and fine lines on the contour of the eye. Two tests were carried out by applying the serum to the contour of the right or left eye, the contour of the other eye being left bare. Furthermore, three tests were carried out by applying a different amount of serum to the contours of the two eyes.

It was observed that the tightening effect varied, with the amount of serum applied, from slight smoothing out of the wrinkles and fine lines to a markedly more visible smoothing/tightening effect. The wrinkles of the crow's foot were thus reduced, as well as those found under the eye, in particular after drying of the film on the skin, while offering a soft, slippery and fresh cosmetic feel.

### EXAMPLE 8

### Evaluation of the Tightening Effect by Measuring with a Dermometer

The composition and the serum of Examples 1 and 2 were tested with a dermometer. This device was described by L. Rasseneur et al. in Influence des Differents Constituants de la Couche Cornée sur la Mesure de son Elasticité [Influence of the Various Constituents of the Horny Layer on the Measurement of its Elasticity], International Journal of Cosmetic Science, 4, 247-260 (1982).

The principle of the method consists in measuring the length of a test specimen of stratum corneum isolated from human skin resulting from a surgical operation, before and after treatment with the test compositions.

To do this, the test specimen is placed between the two jaws of the device, one of which is stationary and the other of which is moveable, in an atmosphere at 30° C. and 40% relative humidity.

Traction is exerted on the test specimen and the curve of the force (in grams) as a function of the length (in millimeters) is recorded, the zero length corresponding to contact between the two clamping jaws of the device.

The tangent to the curve in its linear region is then drawn. The intersection of this tangent with the axis of the abscissa corresponds to the apparent length L₀ of the test specimen at zero force.

The test specimen is slackened and then 2 mg/cm² of the test composition are applied to the stratum corneum. After drying for 15 min, the above stages are again carried out in order to determine the length L₁ of the test specimen after treatment.

The percentage of retraction is defined by:

% retraction=100×(L₁−L₀)/L₀

To characterize a tightening effect, this percentage must be negative and the tightening effect increases in proportion as the absolute value of the percentage of retraction increases.

Results:

The serum according to Example 2 comprising the tightening polymer according to the invention exhibits a percentage of retraction of −1.8±0.4% (mean and standard deviation with regard to 7 samples), whereas the same serum not including tightening polymer gives a percentage of retraction of −0.5±0.3%. This result confirms the tightening effect of the polymer used in this serum and in the composition of Example 1.

For its part, the polymer used in the compositions of Examples 3 and 4 (tested at 12% in ethanol) gives a percentage of retraction of −1.4±0.4% (mean and standard deviation with regard to 7 samples).

This application claims priority to French application 9903531, filed Mar. 22, 1999 the entire contents of which are hereby incorporated by reference.

1. Method for reducing or removing the signs of cutaneous aging, comprising applying onto skin a composition comprising at least one grafted silicone polymer comprising a polysiloxane portion and a portion composed of a non-silicone organic chain, one of the two portions constituting the main chain of the polymer and the other being grafted to the said main chain.

2. Method of tightening the skin comprising applying onto skin a composition comprising at least one grafted silicone polymer comprising a polysiloxane portion and a portion composed of a non-silicone organic chain, one of the two portions constituting the main chain of the polymer and the other being grafted to the said main chain.

3. Method of using in the manufacture of a composition intended to decrease or remove signs of cutaneous ageing, in particular to reduce or remove wrinkles and/or fine lines of the skin and/or to smooth out the skin, of at least one grafted silicone polymer comprising a polysiloxane portion and a portion composed of a non-silicone organic chain, one of the two portions constituting the main chain of the polymer and the other being grafted to the said main chain.

4. Method according to any one of claims 1 to 3, characterized in that said polymer is a polymer with a non-silicone organic backbone grafted by at least one monomer comprising a polysiloxane.

5. Method according to claim 4, characterized in that the said polymer with a non-silicone organic backbone grafted by at least one monomer comprising a polysiloxane is a grafted silicone copolymer comprising:
a) at least one lipophilic monomer (A) with ethylenic unsaturation which is polymerizable by the radical route;
b) at least one polar hydrophilic monomer (B) with ethylenic unsaturation which is copolymerizable with the monomer or monomers of the (A) type such that (A)+(B) varies from 99.99% to 50% by weight; and
c) from 0.01 to 50% by weight of at least one polysiloxane macromer (C) of general formula:
X(Y)ₙSi(R)₃₋ₘZₘ (I)
where:
X denotes a group with ethylenic unsaturation which is copolymerizable with the monomers (A) and (B);
Y denotes a group with divalent bonding;
R denotes a hydrogen, a hydroxyl group, a C₁-C₆ alkyl or alkylamino or alkoxy group or a C₆-C₁₂ aryl group;
Z denotes a polysiloxane unit having a number-average molecular weight of at least 50;
n is 0 or 1 and m is an integer ranging from 1 to 3;
the percentages being calculated with respect to the total weight of the monomers (A), (B) and (C).

6. Method according to claim 5, characterized in that the monomer (A) is chosen from the group composed of n-butyl methacrylate, isobutyl methacrylate, tert-butyl acrylate, tert-butyl methacrylate, 2-ethylhexyl methacrylate, methyl methacrylate, 2-(N-methylperfluorooctanesulphonamido)ethyl acrylate, 2-(N-butylperfluorooctanesulphonamido)ethyl acrylate and their mixtures.

7. Method according to claim 5, characterized in that the monomer (B) is chosen from the group composed of acrylic acid, N,N-dimethylacrylamide, dimethylaminoethyl methacrylate, quatemized dimethylaminoethyl methacrylate, vinylpyrrolidone and their mixtures.

8. Method according to claim 5, characterized in that the polysiloxane macromer (C) has the formula:
with n being a number ranging from 1 to 700.

9. Method according to claim 4, characterized in that the said grafted silicone polymer is capable of being obtained by radical polymerization from the mixture of 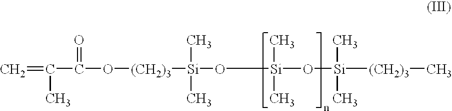
monomers consisting of:
a) 60 weight % of tert-butyl acrylate;
b) 20 weight % of acrylic acid;
c) 20 weight % of silicone macromer of formula (III): 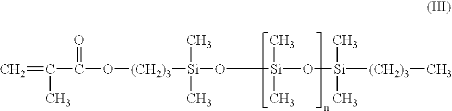
with n being a number ranging from 1 to 700, the percentages by weight being calculated with respect to the total weight of the monomers.

10. Method according to any one of claims 1 to 3, characterized in that the said polymer is a polymer with a polysiloxane backbone grafted by at least one non-silicone organic monomer.

11. Method according to claim 10, characterized in that the said polymer comprises the result of the radical copolymerization between, on the one hand, at least one anionic non-silicone organic monomer exhibiting an ethylenic unsaturation and/or one hydrophobic non-silicone organic monomer exhibiting an ethylenic unsaturation and, on the other hand, a silicone exhibiting, in its chain, at least one functional group capable of reacting with the said ethylenic unsaturations of the said non-silicone monomers with the formation of a covalent bond.

12. Method according to claim 11, characterized in that the said anionic monomer with ethylenic unsaturation is chosen from linear or branched unsaturated carboxylic acids, optionally partially or completely neutralized in the form of a salt, and their mixtures.

13. Method according to claim 12, characterized in that the said unsaturated carboxylic acid is chosen from acrylic acid, methacrylic acid, maleic acid, maleic anhydride, itaconic acid, fumaric acid and crotonic acid.

14. Method according to claim 11, characterized in that the said hydrophobic monomer with ethylenic unsaturation is chosen from alkanol acrylic acid esters and/or alkanol methacrylic acid esters.

15. Method according to claim 14, characterized in that the said hydrophobic monomer with ethylenic unsaturation is chosen from the group composed of isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate, stearyl (meth)acrylate and their mixtures.

16. Method according to claim 10, characterized in that the said silicone polymer comprises, in its structure, the unit of following formula (IV): 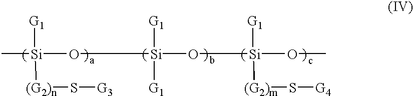
in which the G₁ radicals, which are identical or different, represent hydrogen or a C₁-C₁₀ alkyl radical or alternatively a phenyl radical; the G₂ radicals, which are identical or different, represent a C₁-C₁₀ alkylene group; G₃ represents a polymer residue resulting from the (homo)polymerization of at least one anionic monomer with ethylenic unsaturation; G₄ represents a polymer residue resulting from the (homo)polymerization of at least one hydrophobic monomer with ethylenic unsaturation; m and n are, independently of one another, equal to 0 or 1; a is an integer ranging from 0 and 50; b is an integer which can be between 10 and 350 and c is an integer ranging from 0 and 50, with the proviso that one of the parameters a and c is other than 0.

17. Method according to claim 16, characterized in that the said unit of formula (IV) exhibits at least one, and preferably all, of the following characteristics:
the G radicals denote a C₁-C₁₀ alkyl radical;
n is not zero and the G₂ radicals represent a divalent C₁-C₃ radical;
G₃ represents a polymer radical resulting from the (homo)polymerization of at least one monomer of the carboxylic acid with ethylenic unsaturation type;
G₄ represents a polymer radical resulting from the (homo)polymerization of at least one monomer of the (C₁-C₁₀) alkyl (meth)acrylate type.

18. Method according to claim 17, characterized in that the said grafted silicone polymer corresponding to the formula (IV) is a polydimethylsiloxane to which are grafted, via a connecting link of thiopropylene type, mixed polymer units of the poly((meth)acrylic acid) type and of the poly(alkyl (meth)acrylate) type.

19. Method according to any one of claims 1-3, characterized in that the said grafted silicone polymer represents from 0.03 to 25%, preferably from 0.3 to 6%, better still approximately 2%, of the total weight of the said composition.

20. Composition comprising, in a physiologically acceptable medium, at least one grafted silicone polymer comprising a polysiloxane portion and a portion composed of a non-silicone organic chain, one of the two portions constituting the main chain of the polymer and the other being grafted to the said main chain and one or more plant proteins.

21. Composition comprising, in a physiologically acceptable medium, at least one grafted silicone polymer comprising a polysiloxane portion and a portion composed of a non-silicone organic chain, one of the two portions constituting the main chain of the polymer and the other being grafted to the said main chain and one or more draining, lipolytic, disinfiltrating, slimming, firming, antiglycant and/or vasoprotective compounds.

22. Composition according to claim 20 or 21, characterized in that the said compounds are chosen from: a horse chestnut extract, an ivy extract, a butcher's broom extract, a Bupleurum chinensis extract, an algal extract, caffeine and a rutin derivative.

23. Process for the cosmetic treatment of aged skin, in particular wrinkled skin, comprising the application to the said skin of at least one grafted silicone polymer comprising comprising a polysiloxane portion and a portion composed of a non-silicone organic chain, one of the two portions constituting the main chain of the polymer and the other being grafted to the said main chain in an amount which is effective in softening the wrinkle by a tightening effect.
